(19) United States
(12) Patent Application Publication
(10) Pub. No.: US 2020/0392444 A1
(43) Pub. Date: Dec. 17, 2020

(54) ERYTHROCYTES FOR DRUG DELIVERY

(71) Applicant: POLITECNICO DI MILANO, MILANO (IT)

(72) Inventors: Maria Laura COSTANTINO, MILANO (IT); Giustina CASAGRANDE, MILANO (IT); Monica PIERGIOVANNI, MILANO (IT); Elena BIANCHI, MILANO (IT)

(21) Appl. No.: 16/955,726

(22) PCT Filed: Dec. 20, 2018
(86) PCT No.: PCT/IB2018/060433

§ 371 Date: Jun. 18, 2020

(30) Foreign Application Priority Data
Dec. 21, 2017 (IT) 102017000148368

(51) Int. Cl. C12M 1/00; C12M 3/06; C12M 1/12; C12M 1/36; C12M 1/34

(52) U.S. Cl. CPC C12M 29/26; C12M 23/16; C12M 41/26; C12M 41/48; C12M 25/16

(57) ABSTRACT

The present invention relates to a method and microfluidic device for introducing compounds into red blood cells.

There are described a method for introducing at least one compound into red blood cells, a fluidic circuit for implementing said method, and red blood cells encapsulated according to the method described herein.

### BACKGROUND ART

The loading of compounds into cells, especially red blood cells, is a core step in the research and development of new therapies. The existing technologies aimed at the intracellular administration of compounds are based on electric fields, nanoparticles or chemicals which induce the formation of pores of cell membranes allowing a compound to enter the intracellular region. This method is commonly referred to as “encapsulation” and the cells thus treated are defined as “encapsulated”. However, these methods suffer from numerous complications, including the modification or damage of the compounds to be conveyed, the high mortality of carrier cells, the contact with potentially toxic materials, the poor efficiency.

By way of example, WO2017041050 describes a system for loading cells mediated by the forced passage of said cells through holes with a diameter smaller than the diameter of the cells themselves. W2017008063 describes a microfluidic channel which has a constriction having a lumen less than 4 micrometers, and in any case never more than 90% of the size of the cell used. Through this channel the cells are made to pass, thus creating a deformation on the wall of these cells which leads to the entry of material of interest into the cells following contact with said constriction of the microfluidic channel. In order to operate, the pressure required at the inlet of the device is necessarily higher than 90 Psi, or 6.1 atm.

Casagrande G. et al., in Artif Organs 2016, 40(10): 959-970, describe a method based on the passage of a suspension which comprises the cells to be loaded and the compound in a long glass capillary tube. The results obtained show a poor loading efficiency, together with a high number of echinocytes, indicative of the cellular alteration caused by the same method which is therefore not applicable in clinical practice.

The need of providing an effective method for introducing compounds into cells is strongly felt, where the microfluidic method can manage flow rates such as to make it integrable in a macrofluidic circuit, even in the specific case of biomedical use.

### DESCRIPTION OF THE INVENTION

The present invention relates to a method for introducing compounds into one or more red blood cells, where said method is based on fluid dynamics and diffusion phenomena which, under controlled chemical-physical conditions, surprisingly lead to the temporary opening of pores on parts of the surface of the cell membrane of red blood cells through which said compounds, added to the cell suspension, spread inside the red blood cells.

The present invention further relates to a circuit for implementing said method.

### DESCRIPTION OF THE DRAWINGS

FIG. 1: graph showing, in a logarithmic scale, the relationship between the stress acting on the membrane of the red blood cell and the time for which this stress lasts. The highlighted rectangular area indicates the region which satisfies the conditions of the method according to the present invention.

FIG. 2: diagram of a closed fluidic circuit.

FIG. 3: diagram of an open fluidic circuit.

FIG. 4: A) diagrammatic depiction in longitudinal section of a loading device according to the present invention, loaded with a suspension of red blood cells and a compound; B) diagrammatic depiction of the same longitudinal section and, in the insert, 3D depiction of the loading device.

FIG. 5: fluorescence observed in samples loaded with dextran at increasing concentrations in 58.5 mm long channels, operating at different flow rates.

FIG. 6: cytofluorimetric analysis of loaded samples, in (A) and (B) by varying the length of the channel, in (C), (D), (E) by varying the flow rate.

FIG. 7: confocal microscopy images of red blood cells with and without dextran-fluorescent marker.

FIG. 8: diagram of the machine according to an embodiment.

FIG. 9: diagram of a microfluidic channel, in different embodiments (a-f).

### DETAILED DESCRIPTION OF THE INVENTION

For the purposes of the present invention, the term “compounds” refers to all those materials intended to be introduced, encapsulated into a red blood cell by those skilled in the art. By way of a non-exhaustive example, the compounds are small molecules, peptides, nucleic acids.

The term “encapsulated red blood cells” or “encapsulated blood” means herein the red blood cells loaded with one or more compounds, or the blood whose red blood cells are loaded with one or more compounds.

The present invention first relates to a method for introducing compounds into red blood cells.

Said method comprises:
providing red blood cells from a subject;
providing one or more compounds to be encapsulated in said red cells;
providing a fluidic circuit comprising a device with one or more microfluidic channels;
feeding said device with a suspension comprising said red cells and said one or more compounds;
collecting the red blood cells exiting from said device which are encapsulated red blood cells;
characterized in that:
   said at least one microfluidic channel is made of polymeric material;
   said red blood cells and said one or more compounds are in suspension at a pH between 6.8 and 7.8, preferably between 7.35 and 7.45;
   said at least one microfluidic channel in said device is a conduit having a length I and a section, described by dimensions w and h, said length is comprised between 5 and 500 mm, preferably between 40 and 200, even more preferably between 40 and 130, even more preferably about 60 mm, said section has dimensions such that the smaller dimension of said section is between about 20 and 200 μm; in a preferred embodiment, said section is constant along said conduit, in alternative embodiments, said section varies along the conduit, without however never narrowing below 20 μm; where said device is fed with said suspension so as to obtain an average speed of the fluid comprised between 10⁻⁴ and 10 m/s.

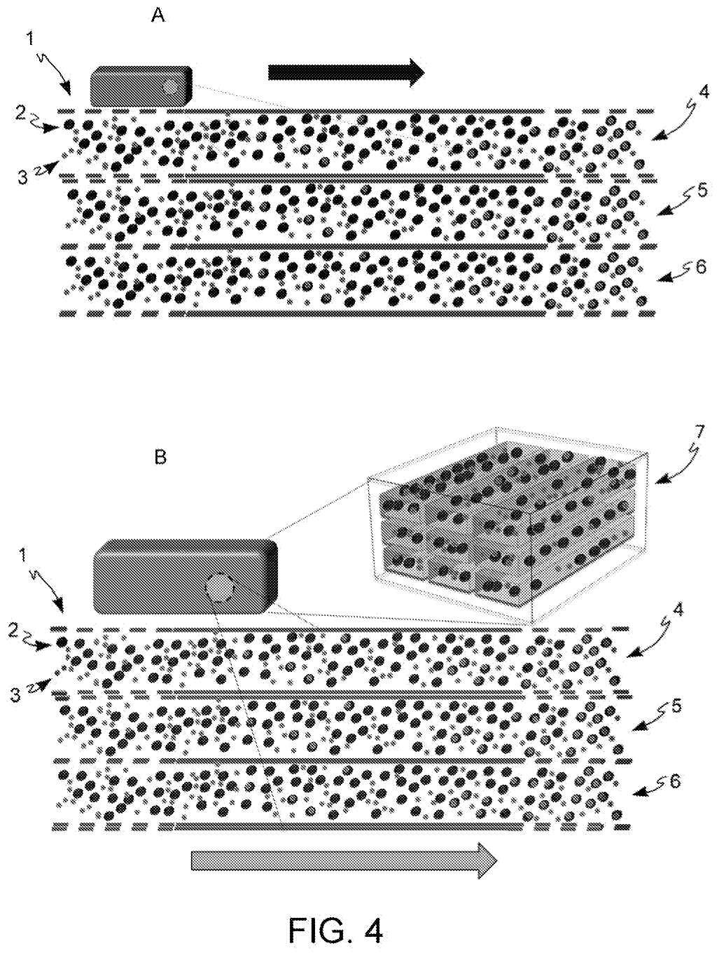

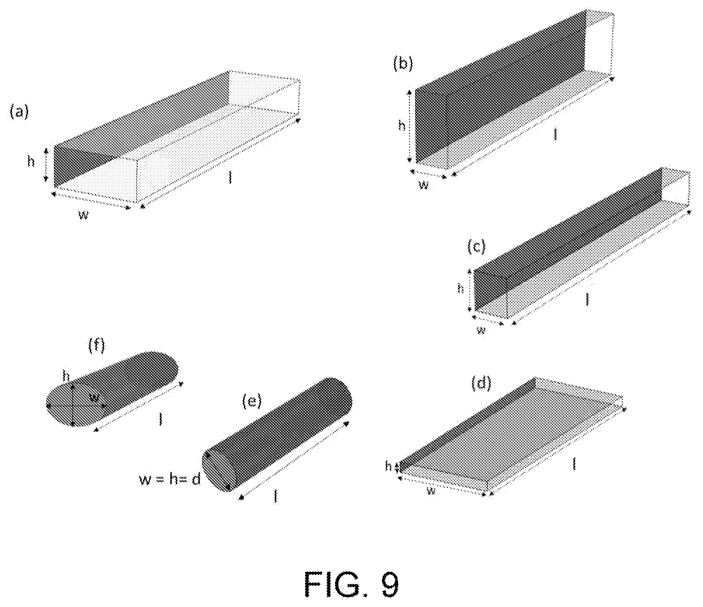

Said microfluidic channel, in its various embodiments schematized in FIG. 9, has a length I between 5 and 500 mm and the smaller dimension (w or h) of the cross section comprised between 20 and 200 μm. As shown in FIG. 9, said microfluidic channel in an embodiment has a rectangular (a, b), or square (c), or extended rectangular (d), or circular (e), oval or ellipsoidal (f) section. Whatever the shape of the section, the dimensions 1, w and h are in the range indicated above. The flow of fluid in said channel, as evidenced by the arrow in FIGS. 4A and 4B, is along the length of said channel. Said smaller dimension of the section belongs to the section of the channel crossed by said fluid. The geometry of the device described herein allows the method according to the present invention to work with load losses of the order of 2.50-3 atm, never more than 5 atm.

The hematocrit of said suspension is between 1 and 50%. In one embodiment, said suspension is obtained in PBS, obtaining a hematocrit of about 1%, or about 5%. In alternative embodiments, the hematocrit of said suspension is between 25 and 40%.

Preferably, albumin is added to said PBS so as to preserve the physiological levels in the blood of the albumin in the suspension, equal to about 5 g/dl.

In an embodiment, said subject is a donor. In an alternative embodiment, said subject is the same patient who needs encapsulated red blood cells.

Alternatively, said suspension also comprises an anticoagulant, e.g. CPD and/or a preservative, e.g. mannitol.

The authors of the present invention have surprisingly shown that, by operating with the method of the present invention, the red blood cells are subjected to a stress field such that on the membrane thereof there occurs the opening of transient pores for a sufficient time to encapsulate the compounds of interest. At the same time, said stress field is not such as to impact the viability of the red blood cells which, after loading, show an excellent viability and conservation of the characteristic biconcave shape. These stresses are defined as sub-hemolytic. Said stress field is generated by the shear stress Tau (τ) in the particular case of laminar motions in circular conduits it is, for example, approximable with the formula (1) and measured in Pascal, Pa=kg/(m*s²).

Σ=4μ Q/(πrᵢ³) (1)

where μ is the viscosity of the fluid, Q the flow rate, and rᵢ the inner radius of the conduit.

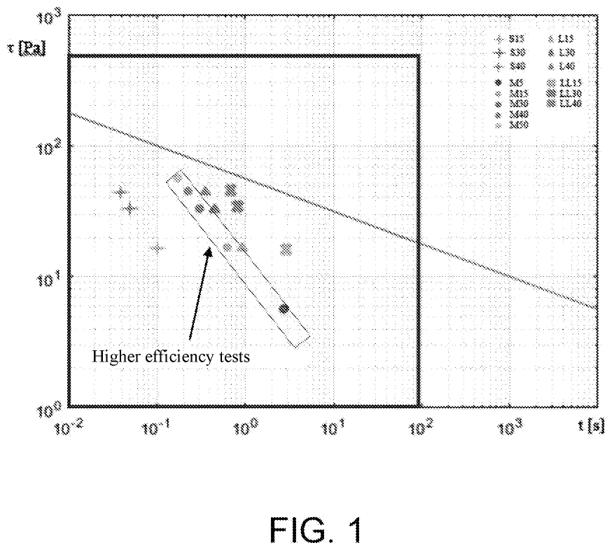

The persistence in time of said shear stress over a minimum time value (tₘᵢₙ) allows the formation of temporary porosity on the membrane of red blood cells. These porosities are reversible if the stress ceases within a maximum time (t_{MAX}). tₘᵢₙ and t_{MAX} vary depending on the mechanical features of each individual's red blood cells, but diagrams are available in the literature statistically showing the probability of hemolysis based on the stress conditions (tau) and the duration of the same stress (t). In the graph in FIG. 1, the line Σ=f(t) represents the shear force τ over time, as proposed by Tillmann W et al., J Biomechanics 1984, 17: 263-279. In the same graph in FIG. 1, the area enclosed in the rectangle is indicative of the shear-time force pairs obtained by operating in the speed ranges according to the present invention, i.e. between 10⁻⁴ and 10 m/s. In particular, operating in such conditions as to remain below the area defined by the line τ=f(t), the integrity of red blood cells is preserved. The shear stress/time pairs included in the rectangular area in FIG. 1 have been surprisingly shown to be such as to allow the loading of the red blood cells without affecting their viability. Shear stresses ranging from 1 to 500 Pa and time between 0.01 and 100 s have been shown capable of solving the technical problem according to the present invention. Even more preferably, operating conditions fall under the straight line τ=f(t) in FIG. 1.

Said microfluidic method, operating with load losses of the order of 2.50-3 atm, never over 5 atm, allows the same to be operated in a macrofluidic circuit, said circuits being able to support the pressures necessary to the method itself. By way of example, the circuit typically used for dialysis is an example of a macrofluidic circuit.

The present invention secondly relates to a fluidic circuit for implementing the method according to the present invention.

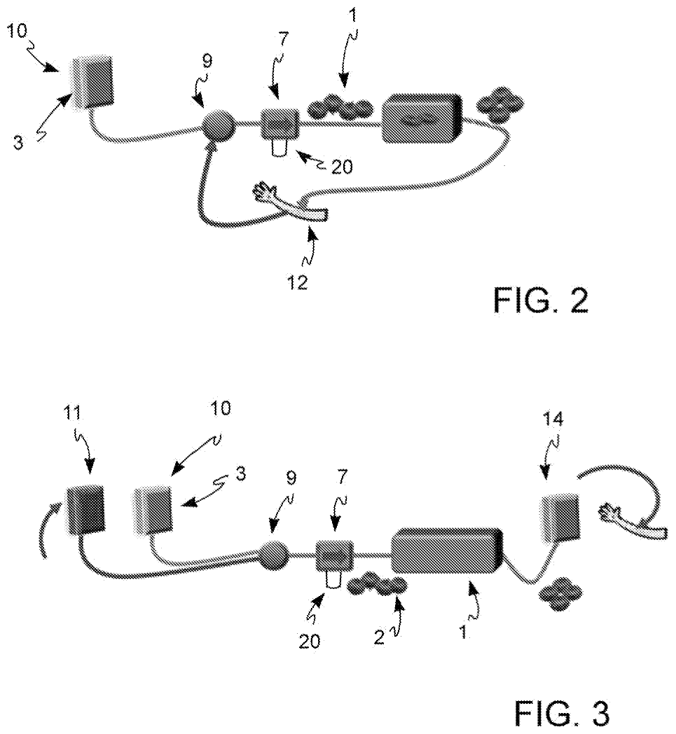

In one embodiment, said circuit is a closed fluid circuit (FIG. 2). In a further embodiment, it is an open fluid circuit (FIG. 3). Said circuit is fed with said at least one compound and with said red blood cells.

With reference to said FIGS. 2 and 3, said circuit comprises: a loading device 1, a pumping device 7, a mixer 9, a control system 20.

Said loading device 1 is made of polymeric material, for example polymethylmethacrylate (PMMA) or poly(dimethylsiloxane) PDMS. Said loading device 1 is a microfluidic device which comprises at least one microfluidic channel, wherein said at least one microfluidic channel is a conduit having a section of such dimensions so that the smaller dimension (w or h) of said section is between about 20 and 200 μm. In a preferred embodiment, said section is constant along the entire length of said channel. In an alternative embodiment, the section varies along said conduit, without however ever narrowing below 20 μm. Said channel has a length l between 5 and 500 mm, preferably between 40 and 200, or between 50 and 130, even more preferably about 60 mm.

FIG. 4A shows a diagram of a portion of said loading device 1 formed by parallel microfluidic channels, 4, 5 and 6, in one embodiment. Said loading device 1 is fed with red blood cells 2, in black and a compound 3, in gray. In the embodiment in FIG. 4B, said device comprises nine parallel microfluidic channels 7 or nine channels 7. Said device is made of polymeric materials.

In alternative embodiments, said loading device comprises at least microchannels. The embodiment which includes thousands of microchannels is particularly preferred. In this embodiment, in each of said microchannels, average speeds are obtained which fall within the range indicated above to obtain the desired loading efficiency and it is possible to load significant volumes of red blood cells, making the loading device suitable for clinical applications, even operating on closed fluid circuits. Purely by way of example, said loading device comprises 25,000 channels and reaches overall capacities between 5 and 300 ml/min.

In one embodiment, said microchannel is a conduit with a circular cross-section, where the diameter of said section is between 20 and 200 μm. In a further embodiment, said microchannel is a conduit with a rectangular section, where the minor side of said rectangle has a shorter length between 20 and 200 μm. In a further embodiment, said conduit has an ellipsoidal section and the minor axis has a length between 20 and 200 μm.

Before being inserted into said loading device 1, said red blood cells and said at least one compound pass through a mixer 9. The mixer is necessary when the flow which is generated in the fluidic circuit according to the present invention is a laminar flow, which therefore does not allow the suspension components to be mixed. The mixer allows improving the contact between the red blood cells and the at least one compound, so as to increase the efficiency of loading.

In the open fluidic circuit embodiment, said mixer 9 receives said at least one compound 3 from a reservoir 10 and said red blood cells 2 from a bag 11. Said bag 11 contains whole blood, or a fraction of the whole blood which comprises red blood cells or, preferably, red blood cells re-suspended in PBS. In said embodiment, said suspension preferably comprises at least one anticoagulant and at least one preservative.

In the closed fluidic circuit embodiment, said mixer 9 receives said at least one compound 3 from a reservoir 10 and said red blood cells 2 in suspension in the whole blood taken from the patient 12. In this embodiment, said at least one compound is preferably re-suspended in PBS, so as to dilute said whole blood entering said mixer.

Said mixer, through said pumping device 7, injects said suspension of at least one compound 3 and red blood cells 2 into said loading device 1. Inside said device, said mixture is distributed in said at least one microchannel and the at least one compound 3 is fed into said red blood cells 2. In the open circuit embodiment, the red blood cells thus processed escape from said loading device 1 and are collected in an encapsulated blood bag 14.

In the closed-circuit embodiment, said encapsulated blood is re-infused into the patient 12.

Said pumping device 7 is selected, for example, from a syringe pump, a peristaltic pump, a centrifugal pump.

Said pumping device 7 is controlled by said control system 20 and said control system 20 imposes in each of said microchannels included in said loading device 1 an average speed between 10⁻⁴ and 10 m/s.

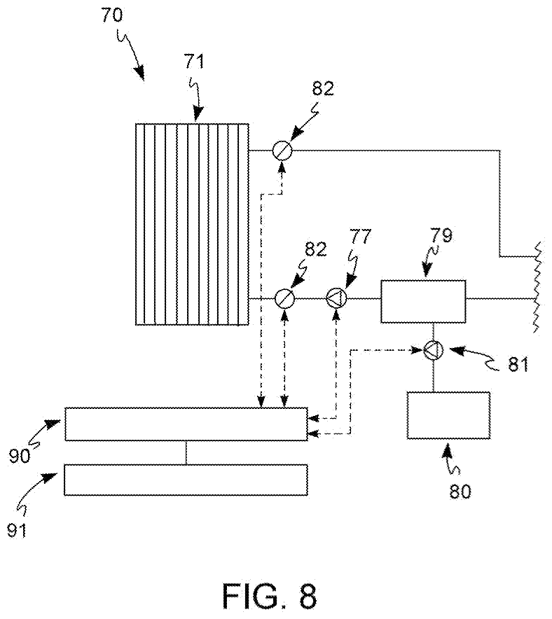

In a further aspect, the present invention relates to a machine 70 for the extracorporeal treatment of the blood which comprises, with reference to the diagram in FIG. 8:

a loading device 71;

at least one pumping device 77 for creating an extracorporeal blood flow between a subject or a blood bag and the loading device 71;

at least one reservoir 80 containing at least one compound to be loaded into said blood;

at least one mixer 79 for mixing said blood with said at least one compound;

optionally, at least one pump 81 for feeding said at least one compound from said reservoir (80) into said mixer (79);

sensors (82) for the control of the chemical-physical parameters of the blood suspension before and/or after passing through said loading device;

a control device (90) for regulating a blood flow value depending on the target value of the blood flow, wherein the control device (90) comprises:

a control/adjustment unit for adapting the current blood flow to the predetermined or selected flow;

optionally, a control/adjustment unit for defining the amount of said at least one compound to be fed into said mixer (79) from said at least one reservoir (80);

an electronic communication unit (91) which is used by the user, on the one hand, to view and, on the other hand, to enter the treatment parameters (corresponding to the parameters of the machine for the treatment of the blood), such as the flow of blood, amount of said at least one compound to be mixed, chemical-physical parameters of the suspension before and/or after passing through said loading device (71). This is done for example through a graphical interface of the machine.

Said machine receives blood from a blood pump which extracts blood from a subject's body through access to the patient, or from a bag containing whole blood or a fraction thereof, preferably a whole blood fraction comprising the red blood cells, more preferably a suspension of red blood cells.

Said loading device (71) is a microfluidic device which comprises at least one microfluidic channel, where said at least one microfluidic channel is a conduit having a section of such dimensions so that the smaller size of said section (described by the dimensions w and h) is comprised between about 20 and 200 μm. Said at least one channel has a length I between 5 and 500 mm, preferably between 40 and 200, or between 50 and 130, even more preferably about 60 mm.

Said control unit (90) regulates the flow rate in each of said microchannels comprised in said loading device so as to have an average speed between 10⁻⁴ and 10 m/s.

Red blood cells loaded according to the present invention can be applied in clinical practice or in experimental research. By way of example, said compounds may be DNA, RNA, monoclonal antibodies, inorganic molecules, organic molecules used for therapeutic purposes, for example in the treatment of tumors or for diagnostic purposes, for example to perform an intracellular labeling.

The method according to the present invention offers a series of advantages with respect to what is available in the prior art.

The loading of red blood cells is mediated by phenomena of fluid dynamics and diffusion. The effect obtained is that of a temporary opening of some of the pores present on the cell membrane of the red blood cells which allows them to enter the same by diffusion of the compound(s) present in the solution in which the red blood cells are found, operating under sub-hemolytic conditions.

Surprisingly, the method, thanks to the identification of a combination of shear stresses/stress times, induces the passage of the compound(s) within the cell without causing any permanent alteration to the physiological state of the membrane. In fact, the porosity is not homogeneously distributed on the membrane of the red blood cells but is located exclusively in the regions characterized by specific shear stresses.

The method according to the present invention also allows avoiding forced contacts with external materials (surfaces of devices, different fluids such as isotonic/hypertonic fluids to be used for loading cells with compounds based on osmosis).

The following examples have the purpose of better clarifying the invention, they are not to be considered in any way limiting the scope of protection.

### Example 1: Encapsulation Efficiency

Blood was collected from 12 healthy donors, subject to informed consent from them. Whole blood was suspended in PBS until a physiological pH solution was obtained. The suspension is centrifuged to separate the red blood cells from the other corpuscular components and from the plasma. The red blood cells are put back in suspension to obtain a final 1% solution of hematocrit. To measure the effectiveness of encapsulation, 40 kDa molecular weight dextran labeled with a fluorophore was used as a compound. Said labeled dextran is added to the suspension until the desired fluorescent molecule concentrations are obtained. As a control (blank), both the same suspension of red blood cells, to which no dextran was added, and the same suspension of red blood cells with dextran (diffusion control) but not inserted into the loading device, thus not fluid-dynamically stressed, were used. The suspension is loaded into a device which comprises: a syringe pump which moves a glass syringe connected with silicone tubes to a straight microchannel with a cross section of 50×50 μm. With the aim of identifying the best experimental conditions, the following parameters were changed: dextran concentration, channel length, flow rate.

Dextran concentrations of 1, 2 or 4 mg/ml, 10, 58.5, 87 or 117 mm long channels and flow rates of 5, 15, 30, 40 or 50 μl/min were used.

After passing into the microchannel, 100 μl of the processed solution were resuspended in PBS, washed and centrifuged to remove the excess dextran left in solution or attached to the outer membrane of the red blood cells and then the dextran encapsulation was measured by fluorescence measurement.

In all the tested configurations there was a significant increase in the fluorescence of cells compared to the blank.

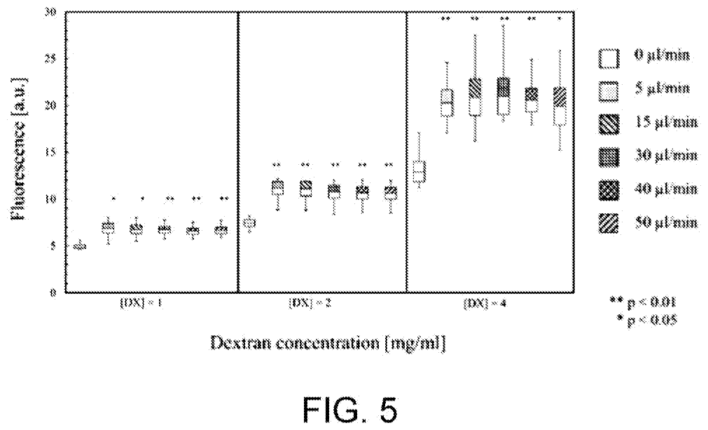

FIG. 5 shows the results obtained in a microchannel with a length of 58.5 mm, at different concentrations of dextran and at different flow rates. By increasing the concentration of dextran, then moving from left to right in FIG. 5, the encapsulation efficiency increases. At all tested capacities, encapsulation is almost constant.

The length of the microchannel significantly influences the encapsulation. Operating with very short microchannels of 10 mm, encapsulation is significant only at dextran concentrations of at least 4 mg/ml and with flow rates of at least 15 μl/min (p value=0.05). Lengths above 58.5 mm do not lead to better encapsulation.

### Example 2: Cell Morphology Analysis after Encapsulation

The samples were analyzed by the flow cytometer to verify the intensity of the fluorescence and the morphology of the cells subjected to the encapsulation process. The fluorescence is evaluated considering the geometric mean of the sample, calculated as (3), where xᵢ is the fluorescence of each cell, n the total number of cells.

$${geo}_{mean} = \frac{log \sum {x}_{i}}{n} \qquad ( 3 )$$

To evaluate the process efficiency, the sample fluorescence is calculated by the comparison with an untreated sample (control, corresponding to diffusive control), taken as a reference, according to the following equation (4):

$$Efficiency ( \% ) = \frac{g e {o}_{mean}^{sample} - g e {o}_{mean}^{control}}{g e {o}_{mean}^{control}} \qquad ( 4 )$$

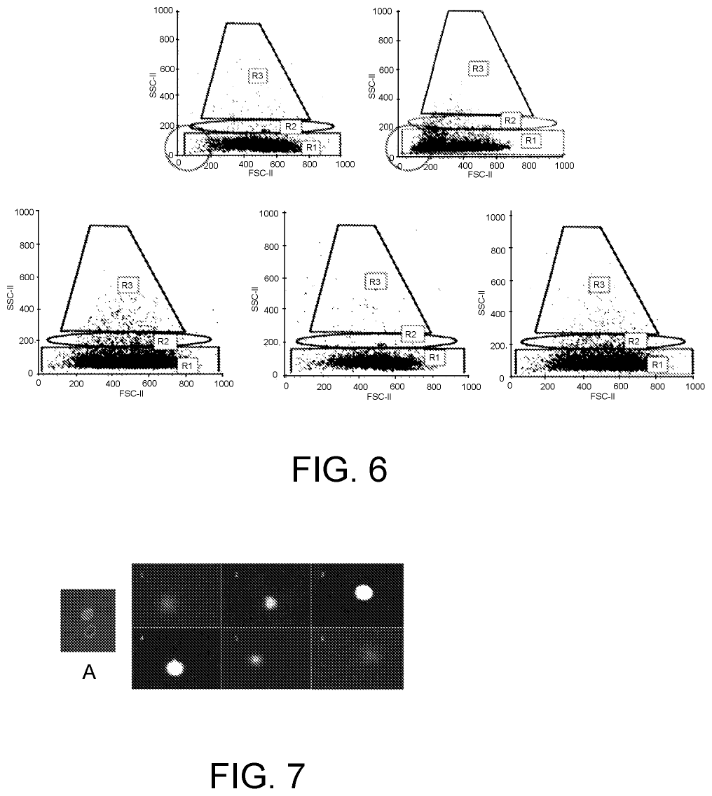

FIG. 6 shows the results obtained after processing the red blood cells, compared with the reference, where the processing is performed with channels of different length (A, B) or by varying the flow rate (C, D, E). In all tested conditions, the largest number of cells is found in the region R1 of the graph, i.e. in the region associated with physiological red blood cells. The echinocytes, or red blood cells altered with a higher level of granularity due to a response to external stimuli, are observable in regions R2 and R3 and remain in a number comparable to the control. The signal moves to areas of smaller size, indicating a large number of cell fragments due to the rupture of red blood cells, in panel B, where the channel is 117 mm long, indicating that the combination of fluidic stress and prolonged time have made the threshold of mechanical hemolysis to be exceeded. This effect is not observed in panels C, D, E, where the length I of the channel is 58.5 mm.

Processed cells were visualized under a confocal microscope. A 12-image stack (Δz=0.8 μm) was acquired to obtain a single cell.

Samples from the test conducted in a 58.5 mm channel with 4 mg/ml of dextran and a flow rate of 30 μl/min were analyzed in fluorescence to confirm the presence of the compound within the cell. Exemplary images are shown in FIG. 7. Panel A shows a red blood cell with FITC-dextran and an unloaded red blood cell. Several optical planes on the same cell confirm the presence of the molecule trapped inside. Red blood cells retain the biconcave disc conformation.

With reference to the graph in FIG. 1, the experimental conditions in which the above experiments were carried out are reported in the graph. In particular, the crosses on the left show the data obtained by working in 10 mm long channels, the dots in 58.5 mm long channels, the triangles in 87 mm long channels and the right crosses are 117 mm channels. The conditions tested herein fall within the preferred area to obtain a good loading efficiency without impact on the viability of red blood cells.

1. A method for introducing compounds into red blood cells which comprises:
providing red blood cells from a subject;
providing one or more compounds to be encapsulated in said red cells;
providing a fluidic circuit which comprises a loading device with one or more microfluidic channels;
feeding said loading device with a suspension comprising said red cells and said one or more compounds;
collecting the red blood cells exiting from said loading device which are encapsulated red blood cells;
characterized in that:
said at least one microfluidic channel is made of polymeric material;
said red blood cells and said one or more compounds are in suspension at a pH between 6.8 and 7.8, preferably between 7.35 and 7.45;
said at least one microfluidic channel is a conduit having a length l parallel to the flow of said suspension in said channel and a section, of dimensions w and h, transverse to the same flow, where said section has dimensions such that the smaller dimension between w and h of said section is between about 20 and 200 μm;
where said loading device is fed with said suspension with a speed comprised between 10⁻⁴ and 10 m/s.

2. The method according to claim 1, wherein said at least one microfluidic channel in said loading device has a length l between 5 and 500 mm, preferably between 40 and 200, or between 50 and 130, even more preferably about 60 mm.

3. The method according to claim 1, wherein said red blood cells are suspended in PBS, or said red blood cells are suspended in whole blood and the hematocrit of said suspension is between 1 and 50%.

4. The method according to claim 3, wherein said red cells are suspended in PBS admixed with albumin.

5. A fluidic circuit for loading red cells with at least one compound, wherein said circuit is an open fluidic circuit comprising: at least one loading device (1), at least one pumping device (7), at least one mixer (9), at least one control system (20), characterized in that said loading device (1) comprises at least one microfluidic channel, wherein said at least one microfluidic channel is a conduit having a section of such dimensions so that the smaller dimension between w and h of said section is between about 20 and 200 μm and said channel has a length l between 5 and 500 mm, preferably between 40 and 200, or between 50 and 130, even more preferably about 60 mm and said control system (20) imposes in each of said microchannels included in said loading device (1) an average speed between 10⁻⁴ and 10 m/s.

6. A machine (70) for extracorporeal blood treatment which comprises:
a loading device (71);
at least one pumping device (77) for creating an extracorporeal blood flow between a subject or a blood bag and the loading device (71);
at least one reservoir (80) containing at least one compound to be loaded into said blood;
at least one mixer (79) for mixing said blood with said at least one compound;
optionally, a pump (81) for feeding said at least one compound from said reservoir (80) into said mixer (79);
sensors (82) for the control of the chemical-physical parameters of the blood suspension before and/or after passing through said loading device;
a control device (90) for regulating a blood flow value depending on the target value of the blood flow, wherein the control device (90) comprises:
a control/adjustment unit for adapting the current blood flow to the predetermined or selected flow;
optionally, a control/adjustment unit for defining the amount of said at least one compound to be fed into said mixer (79) from said at least one reservoir (80);
an electronic communication unit (91) which serves the user, on the one hand, to view and, on the other hand, to enter the treatment parameters,
wherein said loading device (71) is a microfluidic device which comprises at least one microfluidic channel, wherein said at least one microfluidic channel is a conduit having a section, of such dimensions that the smaller dimension of said section is between about 20 and 200 μm and said control unit (90) regulates the flow rate in each of said microchannels included in said loading device so as to have an average speed between 10⁻⁴ and 10 m/s.

7. The machine according to claim 6, wherein said loading device (1, 71) is made of polymeric material, for example polymethylmethacrylate (PMMA) or poly(dimethylsiloxane) PDMS.

8. The machine according to claim 6, wherein said loading device (1, 71) comprises about 25,000 channels and reaches overall capacities between 5 and 300 ml/min.

9. Red blood cells encapsulated with at least one compound according to the method of claim 1.

10. Red blood cells encapsulated according to claim 9 for use in the treatment of pathologies.

11. The circuit according to claim 5, wherein said loading device (1, 71) is made of polymeric material, for example polymethylmethacrylate (PMMA) or poly(dimethylsiloxane) PDMS.

12. The circuit according to claim 5, wherein said loading device (1, 71) comprises about 25,000 channels and reaches overall capacities between 5 and 300 ml/min.
